(19) United States
(12) Patent Application Publication
(10) Pub. No.: US 2021/0162092 A1
(43) Pub. Date: Jun. 3, 2021

(54) MEDICAL HYDROGEL

(71) Applicant: SHANGHAI RUINING BIOTECHNOLOGY CO. LTD, Shanghai (CN)

(72) Inventors: Zhen PAN, Shanghai (CN); Liang CHEN, Shanghai (CN); Sen HOU, Shanghai (CN)

(21) Appl. No.: 17/267,788

(22) PCT Filed: Oct. 23, 2018
(86) PCT No.: PCT/CN2018/111398

§ 371 Date: Feb. 10, 2021

(30) Foreign Application Priority Data
Aug. 10, 2018 (CN) 201810909023.2

(51) Int. Cl. A61L 24/00; A61L 31/14; A61L 24/04; A61L 31/04; A61K 9/06; A61K 47/10; A61K 47/34

(52) U.S. Cl. CPC A61L 24/0031; A61L 31/145; A61L 24/043; A61L 31/041; C08L 79/02; A61K 47/10; A61K 47/34; A61L 2430/40; A61K 9/06

(57) ABSTRACT

A medical hydrogel is formed by in-situ crosslinking an aldehyde-terminated multi-arm star polyethylene glycol and a polyamino compound. The aldehyde group and the multi-arm star polyethylene glycol are linked by a chemical bond such as an ether bond, an amide bond, a urethane bond, an imine bond, or a urea bond. The aldehyde group at the end of the multi-arm polyethylene glycol reacts with the amino group in the polyamino compound to produce Schiff base for crosslinking so that the medical injectable gel is formed. The prepared gel has a short gelling time, a desired gel burst strength, and a good stability in an aqueous solution.

### BACKGROUND

### Technical Field

The present invention relates to the field of biomedical technologies, and specifically, to a medical hydrogel, which can be used for postoperative tissue closure and anti-leakage, anti-tissue adhesion, tissue filler, tissue repair, skin dressing, and drug releasing.

### Related Art

Hydrogel is a soft material containing a large amount of water that is obtained by crosslinking of hydrophilic polymers. It has good physicochemical properties and biological characteristics, such as high water content, high elasticity, softness, and biocompatibility, and has important application value in the biomedical research fields, such as drug delivery and tissue engineering. Injectable hydrogel is a type of hydrogel with a certain fluidity that can be applied by injection. Under external stimuli (changes in temperature, temperature/pH, etc.), the injectable hydrogel presents a phase transition between sol and gel. It is in a liquid state or in a semi-solid state having shear thinning property before being injected into a human body. After being injected into the human body, the injectable hydrogel can gel in situ, and thus no invasive surgeries are required, thereby effectively avoiding the risks of infection, and relieving the pains of patients. Various injectable PEG hydrogels current developed include amphiphilic polyester/polypeptide hydrogels with PEG as a hydrophilic segment, PEG hydrogels prepared by supramolecular interaction, and PEG hydrogels prepared through mild chemical reactions.

Polyethylene glycol (PEG) is a type of non-ionic polymer. Having good biocompatibility and safety, PEG is a synthetic polymer approved by U.S. Food and Drug Administration (FDA) for clinical use in humans. PEG can be used as a pharmaceutical excipient, and PEG with active terminal functional groups can be used to modify drugs (PEGylation). PEGylation technology has a large number of advantages, particularly in terms of the modification of proteins and polypeptide drugs, such as prolonging circulation time in the body, enhancing biological activity, avoiding proteolysis and decreasing immune responses. By connecting active terminal functional groups, such as amino, thiol, azide, alkynyl, and aldehyde, PEG conjugate can be prepared to improve the properties of PEG.

CN105963792A discloses a medical hydrogel composition comprising a first component and a second component, wherein the first component includes polylysine and polyethylenimine; and the second component includes one or more of four-arm polyethylene glycol-succinimidyl glutarate, four-arm polyethylene glycol-succinimidyl succinate and four-arm polyethylene glycol-succinimidyl carbonate. When in use, the nucleophiles (polylysine and polyethylenimine) of the first component may undergo Michael addition reaction with the electrophiles (one or more of four-arm polyethylene glycol-succinimidyl glutarate, four-arm polyethylene glycol-succinimidyl succinate and four-arm polyethylene glycol-succinimidyl carbonate) of the second component, and thus can rapidly form a gel with an excellent low swelling property. However, the succinimidyl ester-terminated PEG material has a very short half-life in water, and is easy to be hydrolyzed. Therefore, it can only be preserved at room temperature in the form of powder for a long period of time by a special technology, and must be used immediately (generally 1 h) after dissolution, making it inconvenient to use.

CN107693838A discloses a medical injectable gel and a preparation method thereof, wherein an aldehyde-terminated hyperbranched polymer HP-PEG-CHO solution with a concentration of 2-20% (w/v) is mixed with a polyamino compound solution with a concentration of 2-20% (w/v) through a two-component syringe and then sprayed. The aldehyde-terminated hyperbranched polymer is crosslinked with the polyamino compound by reacting the aldehyde group with the amino to generate Schiff base, thereby obtaining a medical injectable gel. The aldehyde group in the aldehyde-terminated hyperbranched polymer HP-PEG-CHO is linked to the polymer through ester bonds, and thus the long-term stability in an aqueous solution is relatively low. In addition, the hyperbranched polymer has a wide distribution of molecular weight, and may contain high molecular weight polymers, which is not beneficial to be excreted from human bodies.

### SUMMARY

In view of the shortcomings of the prior art, the present invention provides a medical hydrogel based on a multi-arm star polyethylene glycol that can be stored stably for a long time in an aqueous solution.

The specific technical solutions provided in the present invention are as follows:

A medical hydrogel is provided, which is formed by in-situ crosslinking an aldehyde-terminated multi-arm star polyethylene glycol and a polyamino compound, wherein the aldehyde group and the multi-arm star polyethylene glycol are linked by a bond that is not easy to hydrolyze, such as an ether bond, an amide bond, a urethane bond, an imine bond, or a urea bond.

The polyamino compound is selected from one or more of polyethylenimine and polylysine.

The aldehyde-terminated multi-arm polyethylene glycol is a multi-arm polyethylene glycol with not less than 2 arms and a molecular weight of not less than 2000.

The aldehyde-terminated multi-arm polyethylene glycol has 2-8 arms, and preferably 8 arms.

The aldehyde group is selected from one or more of aromatic aldehydes and alkyl aldehydes, and preferably benzaldehyde.

Another object of the present invention is to provide the use of the medical hydrogel in postoperative tissue closure and anti-leakage, anti-tissue adhesion, tissue filler, tissue repair, skin dressing, and pharmaceutical preparation.

Still another object of the present invention is to provide a method for preparing a medical hydrogel, comprising: dissolving an aldehyde-terminated multi-arm star polyethylene glycol in a pH 4-10 buffer to prepare an aldehyde-terminated multi-arm star polyethylene glycol solution; dissolving a polyamino compound in a pH 4-10 buffer to prepare a polyamino compound solution; and mixing the two solutions to obtain the medical hydrogel.

The aldehyde-terminated multi-arm star polyethylene glycol used in the present invention can be commercially available.

The pH 4-10 buffer is preferably a phosphate buffer or borate buffer with pH 4-10.

The aldehyde-terminated multi-arm star polyethylene glycol solution has a final concentration of 2-30% (w/v), and preferably 10-20% (w/v). The polyamino compound solution has a concentration of 0.5-20%, and preferably 1-5% (w/v).

The molar ratio of the aldehyde group in the aldehyde-terminated multi-arm star polyethylene glycol to the amino in the polyamino compound is 0.01-5:1.

In the specific use of the present invention, a two-component hydrogel is first prepared, which includes a first component containing nucleophilic functional groups and a second component containing electrophilic functional groups. The first component is an aldehyde-terminated hydrophilic compound with not less than two arms. The hydrophilic compound is an aldehyde-terminated multi-arm star polyethylene glycol, and preferably an eight-arm polyethylene glycol (with a molecular weight of 5000-20000). The aldehyde group is one or more of aromatic aldehydes and alkyl aldehydes, and preferably benzaldehyde. The aldehyde group and the polymer may be linked by a chemical bond that is not easy to hydrolyze, such as an ether bond and an amide bond.

The second component may be a polyamino compound, including one or a mixed component of polylysine (including ε-polylysine and poly-L-lysine) and polyethylenimine.

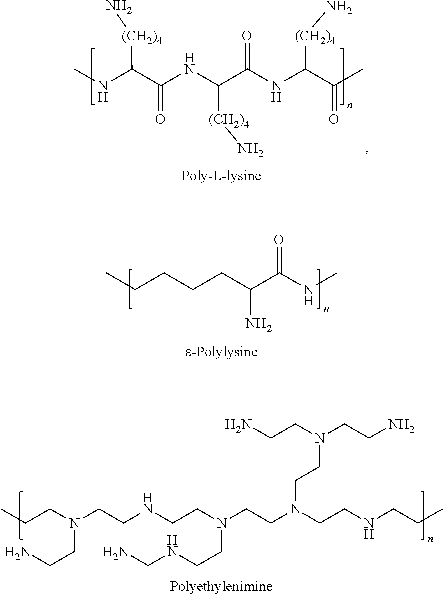

An amide bond-linked benzaldehyde-terminated eight-arm polyethylene glycol, an ether bond-linked benzaldehyde-terminated eight-arm polyethylene glycol, and an ether bond-linked propionaldehyde-terminated eight-arm polyethylene glycol have the chemical structures shown as follows, respectively.

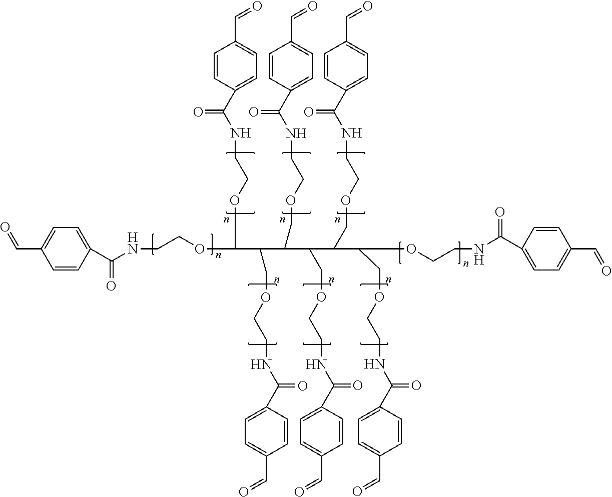

### Amide Bond-Linked Benzaldehyde-Terminated Eight-Arm Polyethylene Glycol

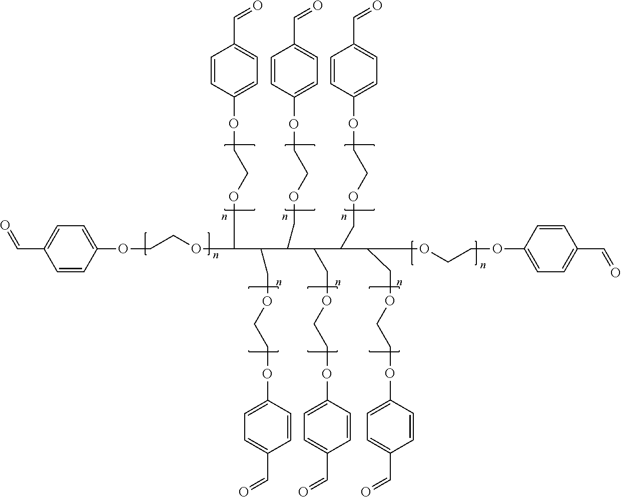

### Ether Bond-Linked Benzaldehyde-Terminated Eight-Arm Polyethylene Glycol

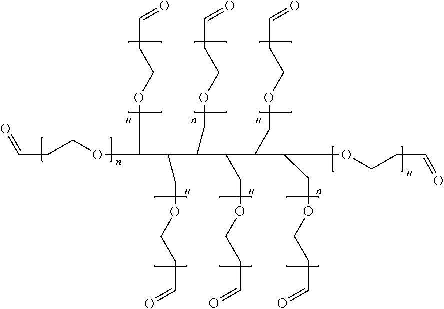

### Ether Bond-Linked Propionaldehyde-Terminated Eight-Arm Polyethylene Glycol

Due to the stability of the aldehyde and amino groups in an aqueous solution, the foregoing two components may be provided in the form of aqueous solution or powder. When in use, the two components are separately dissolved in a buffer, and then the components are mixed to obtain the hydrogel. Alternatively, the two components of the hydrogel may be separately stored in a double-barrel syringe, and when in use, the two components are sprayed or injected to a designated site through a mixing head to form a gel.

In the present invention, the aldehyde group at the end of the multi-arm polyethylene glycol reacts with the amino group in the polyamino compound to produce Schiff base for crosslinking, so that the medical injectable gel is formed. The prepared gel has a short gelling time, a desired gel burst strength, and a good stability in an aqueous solution, and therefore has greater application value than existing medical gels.

### BRIEF DESCRIPTION OF THE DRAWINGS

FIG. 1 shows the observation results of the gelling stability of an ether bond-linked, amide bond-linked, and ester bond-linked aldehyde-terminated polyethylene glycols.

### DETAILED DESCRIPTION

The specific steps of the present invention are described by the following examples, but are not limited to the examples.

The terms used in the present invention, unless otherwise stated, generally have the meanings commonly understood by those of ordinary skill in the art.

The present invention is further described below in detail with reference to specific examples and relevant data. It should be understood that the examples are only used to exemplify the present invention, but do not limit the scope of the present invention in any manner.

In the following examples, various processes and methods that are not described in detail are conventional methods known in the art.

The present invention is further described below with reference to specific examples, but the protection scope of the present invention is not limited to this.

### Example 1

600 mg of an ether bond-linked benzaldehyde-terminated eight-arm polyethylene glycol (8-PEG-O-BA, M.W. 10K) was dissolved in 2 mL of phosphate buffer (pH 7.4) to afford solution A. A solution of 2.2% (w/v) of polyethylenimine in phosphate buffer was prepared as solution B. The solution A and the solution B were mixed in equal volume to obtain a viscous hydrogel with a gelling time of 21 seconds and a gel burst strength of 16 kPa.

### Example 2

600 mg of an ether bond-linked benzaldehyde-terminated eight-arm polyethylene glycol (8-PEG-O-BA, M.W. 13.5K) was dissolved in 2 mL of phosphate buffer (pH 7.4) to afford solution A. A solution of 1.67% (w/v) of polyethylenimine in phosphate buffer was prepared as solution B. The solution A and the solution B were mixed in equal volume to obtain a viscous hydrogel with a gelling time of 22 seconds and a gel burst strength of 11 kPa.

### Example 3

400 mg of an amide bond-linked benzaldehyde-terminated eight-arm polyethylene glycol (8-PEG-amide-BA, M.W. 10K) was dissolved in 2 mL of phosphate buffer (pH 7.4) to afford solution A. A solution of 1.48% (w/v) of polyethylenimine in phosphate buffer was prepared as solution B. The solution A and the solution B were mixed in equal volume to obtain a viscous hydrogel with a gelling time of 2 seconds and a gel burst strength of 13 kPa.

### Example 4

600 mg of an amide bond-linked benzaldehyde-terminated four-arm polyethylene glycol (4-PEG-amide-BA, M.W. 10K) was dissolved in 2 mL of phosphate buffer (pH 7.4) to afford solution A. A solution of 2.2% (w/v) of polyethylenimine in phosphate buffer was prepared as solution B. The solution A and the solution B were mixed in equal volume to obtain a viscous hydrogel with a gelling time of 20 seconds and a gel burst strength of 11 kPa.

### Example 5

400 mg of an amide bond-linked benzaldehyde-terminated eight-arm polyethylene glycol (8-PEG-amide-BA, M.W. 10K) was dissolved in 2 mL of phosphate buffer (pH 7.4) to afford solution A. A solution of 2.44% (w/v) of polylysine in phosphate buffer was prepared as solution B. The solution A and the solution B were mixed in equal volume to obtain a viscous hydrogel with a gelling time of 5 seconds and a gel burst strength of 21 kPa.

### Example 6

400 mg of an amide bond-linked benzaldehyde-terminated eight-arm polyethylene glycol (8-PEG-amide-BA, M.W. 10K) was dissolved in 2 mL of phosphate buffer (pH 7.4) to afford solution A. A solution of 3.66% (w/v) of polylysine in phosphate buffer was prepared as solution B. The solution A and the solution B were mixed in equal volume to obtain a viscous hydrogel with a gelling time of 5 seconds and a gel burst strength of 25 kPa.

### Example 7

600 mg of an ether bond-linked propionaldehyde-terminated eight-arm polyethylene glycol (8-PEG-O-PA, M.W. 10K) was dissolved in 2 mL of phosphate buffer (pH 7.4) to afford solution A. A solution of 1.48% (w/v) of polyethylenimine in phosphate buffer was prepared as solution B. The solution A and the solution B were mixed in equal volume to obtain a viscous hydrogel with a gelling time of 15 seconds and a gel burst strength of 8 kPa.

### Example 8

600 mg of an ether bond-linked benzaldehyde-terminated eight-arm polyethylene glycol (8-PEG-O-BA, M.W. 13.5K) was dissolved in 2 mL of phosphate buffer (pH 7.4) to afford solution A. A solution of 2.75% (w/v) of polylysine in phosphate buffer was prepared as solution B. The solution A and the solution B were mixed in equal volume to obtain a viscous hydrogel with a gelling time of less than 5 minutes and a gel burst strength of 2 kPa.

### Example 9

600 mg of an ether bond-linked benzaldehyde-terminated eight-arm polyethylene glycol (8-PEG-O-BA, M.W. 13.5K) was dissolved in 2 mL of phosphate buffer (pH 7.4) to afford solution A. A solution containing 2.75% (w/v) of polylysine and 1% (w/v) of polyethylenimine (M.W. 1.8K) in phosphate buffer was prepared as solution B. The solution A and the solution B were mixed in equal volume to obtain a viscous hydrogel with a gelling time of 35 seconds and a gel burst strength of 22 kPa.

### Example 10

400 mg of an ester bond-linked benzaldehyde-terminated eight-arm polyethylene glycol (8-PEG-amide-BA, M.W. 10K) was dissolved in 2 mL of phosphate buffer (pH 7.4) to afford solution A. A solution of 1.48% (w/v) of polyethylenimine (M.W. 1.8K) in phosphate buffer was prepared as solution B. The solution A and the solution B were mixed in equal volume to obtain a viscous hydrogel with a gelling time of 5 seconds and a gel burst strength of 13 kPa.

### Example 11

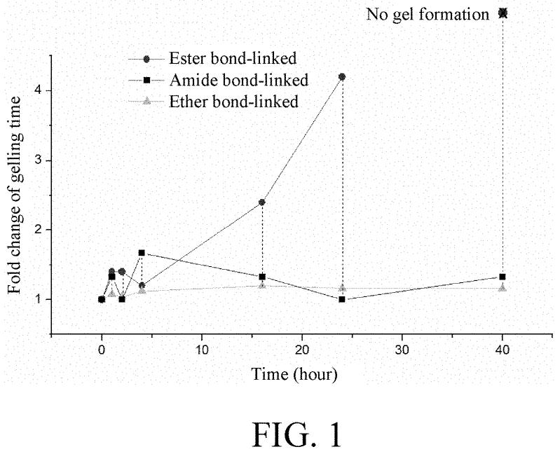

The long-term stabilities of an ether bond-linked, amide bond-linked, and ester bond-linked benzaldehyde-terminated polyethylene glycols in an aqueous solution were compared. To shorten the test time, a basic borate buffer was selected as a solvent to compare the changes in the gelling time at different time points. 400 mg of the ether bond-linked, amide bond-linked, and ester bond-linked benzaldehyde-terminated eight-arm polyethylene glycols (M.W. 10K) were separately dissolved in 2 mL of 0.1M borate buffer (pH 9.2) to afford three solutions A. A solution of 1.48% (w/v) of polyethylenimine (M.W. 1.8K) in phosphate buffer was prepared as solution B. Each of the solutions A was mixed with the solution B in equal volume to obtain a viscous hydrogel. The three hydrogels have initial gelling times of 25 seconds, 2 seconds, and 5 seconds, respectively. The three solutions A were placed in an oven at 37° C. for 1 hour, 2 hours, 4 hours, 16 hours, 24 hours, and 40 hours, and then the differences between the gelling times after mixing with solutions B and the initial gelling times were respectively determined (as shown in FIG. 1). The results showed that the ester bond-linked polyethylene glycol lost gelling capability after 40 hours, whereas the gelling times of the ether bond-linked and the amide bond-linked benzaldehyde-terminated eight-arm polyethylene glycols were basically unchanged.

1. A medical hydrogel, formed by in-situ crosslinking an aldehyde-terminated multi-arm star polyethylene glycol and a polyamino compound, wherein the aldehyde group and the multi-arm star polyethylene glycol are linked by a chemical bond such as an ether bond, an amide bond, a urethane bond, an imine bond, or a urea bond.

2. The medical hydrogel according to claim 1, wherein the polyamino compound is selected from one or more of polyethylenimine and polylysine.

3. The medical hydrogel according to claim 1, wherein the aldehyde-terminated multi-arm star polyethylene glycol is a multi-arm polyethylene glycol with not less than 2 arms and a molecular weight of not less than 2000.

4. The medical hydrogel according to claim 1, wherein the aldehyde-terminated multi-arm star polyethylene glycol has 2-8 arms.

5. The medical hydrogel according to claim 1, wherein the aldehyde group is selected from one or more of aromatic aldehydes and alkyl aldehydes.

6. Use of the medical hydrogel according to claim 1 in postoperative tissue closure and anti-leakage, anti-tissue adhesion, tissue filling, tissue repair, skin dressing, and pharmaceutical preparation.

7. A method for preparing the medical hydrogel according to claim 1, comprising: dissolving the aldehyde-terminated multi-arm star polyethylene glycol in a pH 4-10 buffer to prepare an aldehyde-terminated multi-arm star polyethylene glycol solution; dissolving the polyamino compound in a pH 4-10 buffer to prepare a polyamino compound solution; and mixing the two solutions to obtain the medical hydrogel.

8. The method for preparing the medical hydrogel according to claim 7, wherein the aldehyde-terminated multi-arm star polyethylene glycol solution has a final concentration of 2-30%, and the polyamino compound solution has a concentration of 0.5-20%.

9. The method for preparing the medical hydrogel according to claim 7, wherein the aldehyde-terminated multi-arm star polyethylene glycol solution has a final concentration of 10-20%, and the polyamino compound solution has a concentration of 1-5%.

10. The method for preparing the medical hydrogel according to claim 7, wherein the molar ratio of the aldehyde group in the aldehyde-terminated multi-arm star polyethylene glycol to the amino in the polyamino compound is 0.01-5:1.
